(19) United States
(12) Patent Application Publication
(10) Pub. No.: US 2002/0182590 A1
(43) Pub. Date: Dec. 5, 2002

(54) Determining protein function in cell culture using RNA interference

(75) Inventors: Kevin Strange, Franklin, TN (US); Michael Christensen, Salt Lake City, UT (US); David M. Miller III, Brentwood, TN (US)

Correspondence Address: FULBRIGHT & JAWORSKI L.L.P., SUITE 2400, 600 CONGRESS AVENUE, AUSTIN, TX, 78701-3271, US

(73) Assignee: Vanderbilt University

(21) Appl. No.: 10/155,909

(22) Filed: May 24, 2002

Related U.S. Application Data
(60) Provisional application No. 60/293,830, filed on May 25, 2001.

(51) Int. Cl. C12Q 1/68; C12Q 1/00

(52) U.S. Cl. 435/4; 435/6

(57) ABSTRACT

The present invention addresses one of the major issues in molecular biology today, “functional genomics.” The inventors have provided methods that utilize the phenomenon of RNA interference as a tool for identifying cDNAs that encode proteins with assayable function.

The government owns rights in the present invention pursuant to grant numbers RO1 DK51610 and PO1 DK58212 from the National Institutes of Health. Benefit of priority to copending U.S. Provisional Serial No. 60/293,830, filed May 25, 2001, is claimed, and the content of said application is incorporated herein by reference in its entirety.

### BACKGROUND OF THE INVENTION

A. Field of the Invention

The present invention relates to the fields of molecular biology and nucleic acid biochemistry. More particularly, the invention provides new methods for determining the function of proteins using high-throughput RNA interference screening assays.

B. Related Art

Worldwide efforts to sequence prokaryote, plant and animal genomes began formally in 1990. These efforts have resulted in stunning technological advances and successes. To date, over 20 microbial genomes as well as the genomes for yeast, C. elegans, Drosophila, and Arabidopsis. In addition, the rapid progress of the human genome project also generated an enormous amount of information regarding the sequence of nucleic acids from both normal and abnormal cells.

The total number of expressed human genes has been estimated to be about 100,000, with about 11,000 genes being expressed in any particular cell type (Alberts et al., 1994). These genes can be grouped by their level of expression into abundant, intermediate abundant and rare abundant classes. These classes contain about 4-10 genes, 500 genes, and 11,000 genes respectively, comprising 10%, 40%, and 50% of the total transcripts (Alberts et al., 1994). The majority of expressed genes, therefore, belong to the rare abundant class, and most of the processes for gene identification focus on this category.

In a different kind of analysis, over one million expressed sequence tags (EST) from the human genome have been identified and are listed in the current NCBI dbEST database. Ultimately, most of the expressed genes from human genome will be indexed in the EST database. Maximal use of EST information will greatly accelerate the gene identification process, e.g., using an EST sequence to search the UniGene database to obtain the cluster information for that sequence and to obtain the original plasmids used for EST project for further analysis (Boguski, 1995; Gerhold and Caskey, 1996). The advantage of ESTs is, obviously, that they reflect a subset of genomic sequences that are at least transcribed. By the same token, they are limited by the lack of complete gene information, and in many cases, any functional significance.

Thus, an immediate goal is “to assign some element of function to each of the genes in an organism, and to do this with high-throughput, systematic approaches.” Vukmirovic & Tilghman (2000). However, “currently, a key limiting factor in functional genomics which slows its applications is the lack of fully automated, high-throughput functional profiling technologies to process the increasingly large amounts of raw genomics and differential gene and protein display data.” Novartis Company Research Profile. Thus, what remains, following identification of genomic sequences or ESTs, is the assignment of biological function to them. Clearly, their remains a significant need for those of skill in the art to develop new and more efficient techniques, preferably those with high throughput capability, to assist in these functional analyses.

### SUMMARY OF THE INVENTION

Thus, in accordance with the present invention, there is provided a method for assigning functional properties to polypeptides comprising (a) obtaining a population of double-stranded (ds) RNA molecules, where each unique dsRNA molecule is segregated from other members of the dsRNA population; (b) contacting one or more members of said dsRNA population with a host cell; and (c) measuring one or more phenotypic parameters of the host cell of step (b), wherein a change in a phenotypic parameter in the host cell of step (b), as compared to a host cell not contacted with dsRNA, assigns to a polypeptide or polypeptides encoded by the one or more members of said dsRNA population of step (b) a functional property.

The method may further comprise (d) contacting each of the dsRNA sequences used in step (b) with a different host cell and (e) measuring one or more phenotypic parameters of each of the host cells of step (d), wherein a change in a phenotypic parameter a host cell of step (d), as compared to a host cell not contacted with dsRNA, assigns to a polypeptide encoded by the dsRNA of step (d) a functional property. The method may also further comprise the step, prior to step (a), of preparing dsRNA, and even prior to the step of preparing dsRNA, a step of preparing cDNA. The method may also comprise DNA or RNA subtraction or sequencing of one or more nucleic acids following assignment of a functional property.

Step (b) may comprise contacting a first set of about 10 to about 100 different dsRNA sequences with said host cell. The host cell may be bacterial, yeast, or mammalian. The dsRNA population may be derived from a CDNA library or DNA templates generated using known gene sequence and PCR methods. The method may comprise concurrent testing of multiple sets of about 10 to about 100 dsRNA with individual host cells.

In another embodiment, there is provided a method for identifying a functionally relevant polypeptides comprising (a) obtaining a population of double-stranded (ds) RNA molecules, where each unique dsRNA molecule is segregated from other members of the dsRNA population; (b) contacting one or more members of said dsRNA population with a host cell; and (c) measuring one or more phenotypic parameters of the host cell of step (b), wherein a change in a phenotypic parameter in the host cell of step (b), as compared to a host cell not contacted with dsRNA, assigns to a polypeptide or polypeptides encoded by said one or more members of said dsRNA population of step (b) a relevant function.

In yet another embodiment, there is provided a method for screening a population of nucleic acids for functionally relevant polypeptides comprising (a) obtaining a population of double-stranded (ds) RNA molecules, where each unique dsRNA molecule is segregated from other members of the dsRNA population; (b) contacting one or more members of said dsRNA population with a host cell; and (c) measuring one or more phenotypic parameters of the host cell of step (b), wherein a change in a phenotypic parameter in the host cell of step (b), as compared to a host cell not contacted with dsRNA, assigns to a polypeptide or polypeptides encoded by said one or more members of said dsRNA population of step (b) a relevant function.

### BRIEF DESCRIPTION OF THE DRAWINGS

The following drawings form part of the present specification and are included to further demonstrate certain aspects of the present invention. The invention may be better understood by reference to these drawings and the detailed description presented below.

FIG. 1.—RNA interference in cultured unc-4::GFP-expressing cholinergic motor neurons. Isolated blastomeres were treated immediately after plating with 10 μg/ml GFP dsRNA. Cells were scored as exhibiting bright, medium or dim fluorescence. Five to seven random fields (2,000-3,000 cells) in each culture were imaged on successive days after initial dsRNA treatment. The ratio of GFP-expressing cell to total cells was quantified in each field. Values are means±S.D. (n=2 experiments).

FIGS. 2A and 2B—Patch clamp recordings from a cultured C. elegans neuron. FIG. 2A shows whole-cell currents elicited by stepping membrane potential from −100 to +100 mV. FIG. 2B shows current-to-voltage relationship for peak and steady-state currents shown in FIG. 2A.

FIGS. 3A-3C—Whole-cell recordings from C. elegans body wall muscles. FIG. 3A shows whole-cell currents elicited by stepping membrane from −100 mV to +100 mV. Holding potential =−80 mV. FIG. 3B shows steady-state current-to-voltage relationship. Values are means±S.E. (n=8). FIG. 3C shows inhibitory effects of bath addition to 20 mM TEA or 20 mM TEA and 3 mM 4-aminopyridine (4-AP). Steady-state currents in the presence and absence of the drugs were measured at +80 mV. Drug effects were reversible. Values are means±S.E. (n=3-5).

FIGS. 4A-4C—Effect of GFP dsRNA on GFP levels in myo-3::GFP-expressing muscle cells. FIG. 4A shows the relative number of myo-3::GFP-expressing cells in cultures treated with GFP dsRNA. GFP fluorescence in single cells was scored as bright, medium, or dim. Data is based on fluorescence micrographs. Values are means±SD of two independent experiments. FIG. 4B shows the pixel intensities (relative to control) of images of myo-3::GFP-expressing cells treated with dsRNA for 1-3 days. Images were obtained daily for three successive days from 16 random fields visualized in paired control and dsRNA-treated cell cultures. Values are means±SD of two independent experiments. FIG. 4C shows the total number of muscle cells and neurons present in micrographs obtained from control and dsRNA-treated cell cultures. Cells were exposed to dsRNA immediately after plating. Imaging protocol was the same as described in FIG. 4A. Values are means±SD of two independent experiments.

FIGS. 5A-5B—Effect of dsRNA on Gene Expression in Cultured Neurons. FIG. 5A shows pixel intensities (relative to control) of images of unc-119::GFP-expressing cells treated with dsRNA for 1-5 days. Images were obtained from 16 random fields visualized in paired control and dsRNA-treated cell cultures. FIG. 5B shows the relative number of unc-4::GFP-expressing neurons in cultures treated with GFP dsRNA. GFP fluorescence in single cells was scored as bright, medium, or dim. Imaging protocol was similar to that described in FIG. 5A. Values are means±SD of 2-3 experiments.

### DETAILED DESCRIPTION OF THE INVENTION

Genome sequencing has revolutionized molecular biology. In its wake, a new field referred to as “functional genomics” has emerged as one of the most active areas of research. Functional genomics involves the assignment of biological function to newly identified genes and, importantly, the elucidation of the organization, integration and control of proteins as part of a larger network controlling biological processes. It is widely recognized that the significant challenges posed by attempts to define the genetic basis of biological function necessitates the study of experimentally more manipulable “model organisms.” Cowley (1999); Hodgkin et al. (1995); Kao (1999).

In a broad sense, suitable model organisms are those that provide an experimental platform for examining a biological problem. In the context of functional genomics, this term has a more distinct meaning. Model organisms must be “simple” organisms that provide unique experimental advantages for defining gene function. These advantages include a short life cycle, cellular and molecular manipulability, and susceptibility to straightforward and rapid genetic analysis of even complex physiologic processes.

The present invention addresses this important problem in the field of functional genomics by developing methods for high-throughput screening of gene function in cultured cells of C. elegans, as well as other non-vertebrate model organisms.

A. RNA Interference

RNA interference (RNA₁) is a form of gene silencing triggered by double-stranded RNA (dsRNA). DsRNA activates post-transcriptional gene expression surveillance mechanisms that appear to function to defend cells from virus infection and transposon activity. Fire et al. (1998); Grishok et al. (2000); Ketting et al. (1999); Lin & Avery (1999); Montgomery et al. (1998); Sharp (1999); Sharp & Zamore (2000); Tabara et al. (1999). Activation of these mechanisms targets mature, dsRNA-complementary mRNA for destruction. RNA₁ offers major experimental advantages for study of gene function. These advantages include a very high specificity, ease of movement across cell membranes, and prolonged down-regulation of the targeted gene. Fire et al. (1998); Grishok et al. (2000); Ketting et al. (1999); Lin & Avery (1999); Montgomery et al. (1998); Sharp (1999); Sharp & Zamore (2000); Tabara et al (1999). Moreover, dsRNA has been shown to silence genes in a wide range of systems, including plants, protozoans, fungi, C. elegans, Trypanasoma and Drosophila. Grishok et al. (2000); Sharp (1999); Sharp & Zamore (1999).

Interestingly, RNAᵢ can be passed to progeny, both through injection into the gonad or by introduction into other parts of the body (including ingestion) followed by migration to the gonad. Several principles are worth note (see Plasterk & Ketting, 2000) First, the dsRNA should be directed to an exon, although some exceptions to this rule have been shown. Second, a homology threshold (probably about 80-85% over 200 bases) is required. Most tested sequences are 500 base pairs or greater. Third, the targeted mRNA is lost after RNA₁. Fourth, the effect is non-stoichometric, and thus incredibly potent. In fact, it has been estimated that only a few copies of dsRNA are required to knock down >95% of targeted gene expression in a cell. Fire et al. (1998).

Although the precise mechanism of RNA₁ is still unknown, the involvement of permanent gene modification or the disruption of transcription have been experimentally eliminated. It is now generally accepted that RNA₁ acts post-transcriptionally, targeting RNA transcripts for degradation. It appears that both nuclear and cytoplasmic RNA can be targeted. Bosher and Labouesse (2000).

B. The Present Invention

Using RNA₁, the inventors have demonstrated that one can rapidly identify genes responsible for any biological process for which an assay is available. The method is applied “blind” in the sense that one will interrogate a large number of undefined genetic targets. Such a process involves the production of a DNA template library from a suitable organism using standard procedures. DNA templates are then used to transcribe dsRNA. Cultured cells are treated with single dsRNAs and functional assays applied, optionally utilizing automated procedures. Disruption of function, as measured by the assay, indicates that the dsRNA has eliminated the expression of the cognate mRNA encoding a protein required for the specific function being assayed. The identity of the gene encoding the mRNA is automatically known if the dsRNA is synthesized from DNA templates produced using primers designed from genome sequence. For dsRNA produced from cDNA libraries sequence information is obtained from the corresponding cDNA. The corresponding gene is then identified by BLAST searching of appropriate genomic databases.

C. cDNA and DNA Template Library Production

In one embodiment, the present invention provides for the generation of a cDNA library. Messenger RNA is isolated from a selected cell line or organism using phenol/chloroform extraction and isopropanol precipitation. cDNA is synthesized by randomly primed reverse transcriptase polymerase chain reaction (RT-PCR), ligated into Lambda Zap II (Stratagene) and packaged into phage particles with Gigapack III packaging extract (Stragene). A typical high quality cDNA library will contain 10⁶-10⁷ independent clones.

The cDNA library is plated at low density and individual plaques isolated and suspended in phage buffer. Aliquots of single phage suspensions are transferred to 96-well microtitre plates for polymerase chain reaction (PCR). Individual cDNA inserts are amplified by PCR using T3 and T7 primer sequence. Automated procedures may be used to aid this time intensive procedure. The separated cDNA library is designated as a “DNA template library.”

A DNA template library may also be produced by utilizing genome sequence information. Primer pairs containing T7 polymerase sequence are generated for each identified gene in the organism's genome. DNA templates are synthesized by RT-PCR from total organism mRNA or by PCR from cosmid vectors containing sequenced genomic DNA.

D. dsRNA Production

dsRNA is synthesized using well-described methods (Fire et al, 1998). Briefly, sense and antisense RNA are synthesized from DNA templates using T7 polymerase (MEGAscript, Ambion). After the synthesis is complete, the DNA template is digested with DNaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedures may be used to aid this time intensive procedure. The sum of the individual dsRNA species is designated as a “dsRNA library.”

E. Model organism cell culture

RNA interference has been shown to be a potent mechanism for disrupting gene expression in plants, protozoans and numerous invertebrate animals. Cultured cells derived from these organisms will provide the experimental platform or “host system” for defining gene function.

Cell culture for nematodes has not been widely available. However, the inventors have recently made substantial progress in culturing cells from C. elegans. N2 adult nematodes are grown on 10 cm NGM enriched peptone plates and harvested just prior to overcrowding. Eggs are isolated by treating adults with a solution of bleach (25% Clorox) and NaOH (0.5 M) until 50% of the adults are lysed. The whole worm lysates are then washed three times in quick succession with egg buffer (118 mM NaCl, 48 mM KCl, 2 mM CaCl₂, 2 mM MgCl₂, 25 mM HEPES, pH=7.4, 345 mOsm). Following the third wash, eggs are separated from the dead carcasses and debris by density centrifugation in a 30% sucrose solution (400×g for 3 minutes).

In a sterile tissue culture laminar flow hood, the floating “egg layer” is harvested using a transfer pipette, diluted 5 fold with sterile H₂O, and centrifuged (400×g). The pellet, which is comprised mostly of mixed stage embryos and un-hatched larvae, is then resuspended in 0.5 ml (per 10 cm dish) of egg buffer containing 1 U/ml chitinase (Sigma, ˜1000 U/mg) and allowed to incubate for 30 minutes at room temperature with occasional gentle agitation. The reaction is halted by the addition of L-15 tissue culture medium (Gibco, adjusted to 345 mOsm using sucrose) containing 10% fetal bovine serum, penicillin (50 U/ml), and streptomycin (50 μg/ml). This medium is designated “L-15-10.”

Cells are washed once in L-15-10 and then resuspended this media at a concentration of 5,000 cells/microliter. The suspended cells are filtered through a 5-micron syringe filter (Millipore). Filtered cells are seeded onto acid-washed glass cover coated with peanut agglutinin (Sigma, 1 mg/ml in dH₂O). Cell cultures are maintained at 24° C. in a humidified cell culture incubator.

F. Assay Formats

Functional assays are categorized in four groups: 1) native cell functions, 2) bacterial and viral infection, 3) heterologous expression, and 4) mechanisms of drug and toxin action.

1. Native Cell Functions

The basic cellular functions of model organisms are the same as those of other eukaryotes. Defining the genes that mediate cellular processes is one of the defining goals of the field of functional genomics. A host of native cell functions can be readily assayed using available or easily developed technology. For example, ion transport processes can be studied using imaging methods and fluorescent probes that track membrane voltage or intracellular concentration of ions such as Ca²⁺, Na⁺, K⁺, Cl⁻ and H⁺. Cell proliferation and programmed cell death is readily quantified using commercially available (Molecular Probes), fluorescence-based live/dead, proliferation and cell cycle assays. The genes that cells utilize to survive stresses such as osmotic, oxidative and heat shock can be readily assessed using fluorescence-based live/dead assays.

2. Heterologous Expression

Model organism cells can be readily engineered to express vertebrate and other foreign genes. When expressed in non-native cell types, foreign genes will frequently recapitulate their native cellular functions. For example, a heterologously expressed neurotransmitter receptor may associate with signaling molecules such as G proteins, phosphatases, kinases, etc. Application of a neurotransmitter to the cell heterologously expressing the receptor will therefore activate signaling cascades and functional responses similar to those of the native cell type.

The functional responses associated with heterologous expression of a vertebrate gene in a model organism cell can be assayed. For example, if activation of a vertebrate receptor triggers increases in intracellular Ca²⁺ in a model organism cell, those Ca²⁺ signals can be readily measured using imaging methods and the fluorescent dye fura-2 in the presence of RNA interference. It is then possible to identify the genes responsible for the signaling events leading to the Ca²⁺ increase.

3. Bacterial and Viral Infection

Identification of the genes utilized by host cells to defend themselves against bacteria and viruses, as well as the host genes that pathogens exploit in the infection process, represents a fundamentally important area of biomedical research with broad practical applications. RNA interference screening in model organism cell cultures represents a powerful approach to rapidly identify these genes. Two types of functional assays will be employed to define the genetic basis of cellular infection and defense. Commercially available (Molecular Probes), fluorescence-based live/dead assays will allow identification of host cell genes required for the infection process. Similarly, quantification of bacterial cell viability will allow identification of the genes involved in the defense of cells against a pathogen.

The infection process can also be quantified by engineering pathogens with a green fluorescent protein (GFP) reporter cassette. Infection is then quantified by monitoring changes in GFP expression in the host cells. Host cells can also be engineered to express GFP reporters that are activated during infection.

4. Mechanisms of Drug and Toxin Action

The effects of drugs and toxins on cells are critically dependent on the presence and functioning of specific cellular proteins. Model organism cell culture combined with dsRNA screening will allow the rapid identification of genes that encode these proteins. The genes required for the action of specific toxins will be assessed using commercially available (Molecular Probes), fluorescence-based live/dead assays. Disruption of the expression of a gene required for toxin action will result in increased cell survival. Conversely, disruption of genes required for detoxifying specific agents will result in increased cell death.

Assays to identify genes required for drug action will be based on the overall effect of the drug on a given cellular phenotype. For example, if a drug inhibits cellular proliferation, cell growth will be assessed using fluorescence-based proliferation assays. Drugs that alter gene expression, ion transport and cellular metabolism can be assayed using fluorescent reporter genes, fluorescent probes that track the intracellular concentration of ions such as Ca²⁺, Na⁺, K⁺, Cl⁻ and H⁺, and endogenous fluorescent signals that change in response to metabolites such as NADH.

5. Assay Protocol

Cells from specific model organisms will be cultured in multi-well format culture plates. Thirty μg of a specific dsRNA is added to 1 ml of culture medium covering the cells. After a 2 hr incubation, an additional 2 ml of culture medium is added to each cell culture. Functional assays are performed 2 days later on control and dsRNA-treated cells.

G. Primers and Probes

1. Primer Design

The term primer, as defined herein, is meant to encompass any nucleic acid that is capable of priming the synthesis of a nascent nucleic acid in a template-dependent process. Typically, primers are oligonucleotides from ten to twenty-five base pairs in length, but longer sequences can be employed. Primers may be provided in double-stranded or single-stranded form, although the single-stranded form is preferred.

2. Oligonucleotide Synthesis

Oligonucleotide synthesis is performed according to standard methods. See, for example, Itakura and Riggs (1980). Additionally, U.S. Pat. Nos. 4,704,362; 5,221,619; 5,583,013; each describe various methods of preparing synthetic structural genes.

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al, 1978; Gillam et al., 1979). Under controlled conditions, polynucleotide phosphorylase adds predominantly a single nucleotide to a short oligodeoxynucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic DNA synthesizers.

Phosphoramidite chemistry (Beaucage and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

H. Polymerases

1. Reverse Transcriptases

According to the present invention, a variety of different reverse transcriptases may be utilized. The following are representative examples.

M-MLV Reverse Transcriptase. M-MLV (Moloney Murine Leukemia Virus Reverse Transcriptase) is an RNA-dependent DNA polymerase requiring a DNA primer and an RNA template to synthesize a complementary DNA strand. The enzyme is a product of the pol gene of M-MLV and consists of a single subunit with a molecular weight of 71 kDa. M-MLV RT has a weaker intrinsic RNase H activity than Avian Myeloblastosis Virus (AMV) reverse transcriptase which is important for achieving long full-length complementary DNA (>7 kB).

M-MLV can be use for first strand cDNA synthesis and primer extensions. Storage recommend at −20° C. in 20 mM Tris-HCl (pH 7.5), 0.2M NaCl, 0.1 mM EDTA, 1 mM DTT, 0.01% Nonidet® P-40, 50% glycerol. The standard reaction conditions are 50 mM Tris-HCl (pH 8.3), 7 mM MgCl₂, 40 mM KCl, 10 mM DTT, 0.1 mg/ml BSA, 0.5 mM ³H-dTTP, 0.025 mM oligo(dT)₅₀, 0.25 mM poly(A)₄₀₀ at 37° C.

M-MLV Reverse Transcriptase, RNase H Minus. This is a form of Moloney murine leukemia virus reverse transcriptase (RNA-dependent DNA polymerase) which has been genetically altered to remove the associated ribonuclease H activity (Tanese and Goff, 1988). It can be used for first strand cDNA synthesis and primer extension. Storage is at 20° C. in 20 mM Tris-HCl (pH 7.5), 0.2M NaCl, 0.1 mM EDTA, 1 mM DTT, 0.01% Nonidet® P-40, 50% glycerol.

AMV Reverse Transcriptase. Avian Myeloblastosis Virus reverse transcriptase is a RNA dependent DNA polymerase that uses single-stranded RNA or DNA a a template to synthesize the complementary DNA strand (Houts et al, 1979). It has activity at high temperature (42° C.-50° C.). This polymerase has been used to synthesize long cDNA molecules.

Reaction conditions are 50 mM Tris-HCl (pH 8.3), 20 mM KCl, 10 mM MgCl₂, 500 μM of each dNTP, 5 mM dithiothreitol, 200 μg/ml oligo-dT₍₁₂₋₁₈₎, 250 μg/ml polyadenylated RNA, 6.0 pMol ³²P-dCTP, and 30 U enzyme in a 7 μl volume. Incubate 45 min at 42° C. Storage buffer is 200 mM KPO₄ (pH 7.4), 2 mM dithiothreitol, 0.2% Triton X-100, and 50% glycerol. AMV may be used for first strand cDNA synthesis, RNA or DNA dideoxy chain termination sequencing, and fill-ins or other DNA polymerization reactions for which Klenow polymerase is not satisfactory (Maniatis et al., 1976).

2. DNA polymerases

The present invention also contemplates the use of various DNA polymerase. Exemplary polymerases are described below.

Bst DNA Polymerase, Large Fragment. Bst DNA Polymerase Large Fragment is the portion of the Bacillus stearothermophilus DNA Polymerase protein that contains the 5′→3′ polymerase activity, but lacks the 5′→3′ exonuclease domain. BST Polymerase Large Fragment is prepared from an E. coli strain containing a genetic fusion of the Bacillus stearothermophilus DNA Polymerase gene, lacking the 5′→3′ exonuclease domain, and the gene coding for E. coli maltose binding protein (MBP). The fusion protein is purified to near homogeneity and the MBP portion is cleaved off in vitro. The remaining polymerase is purified free of MBP (Iiyy et al, 1991).

Bst DNA polymerase can be used in DNA sequencing through high GC regions (Hugh & Griffin, 1994; McClary et al., 1991) and Rapid Sequencing from nanogram amounts of DNA template (Mead et al., 1991). The reaction buffer is 1× ThermoPol Butter (20 mM Tris-HCl (pH 8.8 at 25° C.), 10 mM KCl, 10 mM (NH₄)₂SO₄, 2 mM MgSO₄, 0.1% Triton X-100). Supplied with enzyme as a 10× concentrated stock.

Bst DNA Polymerase does not exhibit 3′→5′ exonuclease activity. 100 μ/ml BSA or 0.1% Triton X-100 is required for long term storage. Reaction temperatures above 70° C. are not recommended. Heat inactivated by incubation at 80° C. for 10 min. Bst DNA Polymerase cannot be used for thermal cycle sequencing. Unit assay conditions are 50 mM KCl, 20 mM Tris-HCl (pH 8.8), 10 mM MgCl₂, 30 nM M13mp18 ssDNA, 70 nM M13 sequencing primer (-47) 24 mer (NEB #1224), 200 μM daTP, 200 μM dCTP, 200 μM dGTP, 100 μM ³H-dTTP, 100 μg/ml BSA and enzyme. Incubate at 65° C. Storage buffer is 50 mM KCl, 10 mM Tris-HCl (pH 7.5), 1 mM dithiothreitol, 0.1 mM EDTA, 0.1% Triton-X-100 and 50% glycerol. Storage is at −20° C.

VENT_{R}® DNA Polymerase and VENT_{R}® (exo⁻) DNA Polymerase. Vent_{R} DNA Polymerase is a high-fidelity thermophilic DNA polymerase. The fidelity of Vent_{R} DNA Polymerase is 5-15-fold higher than that observed for Taq DNA Polymerase (Mattila et al., 1991; Eckert and Kunkel, 1991). This high fidelity derives in part from an integral 3′→5′ proofreading exonuclease activity in Vent_{R} DNA Polymerase (Mattila et al., 1991; Kong et al., 1993). Greater than 90% of the polymerase activity remains following a 1 h incubation at 95° C.

Vent_{R} (exo−) DNA Polymerase has been genetically engineered to eliminate the 3′→5′ proofreading exonuclease activity associated with Vent_{R} DNA Polymerase (Kong et al., 1993). This is the preferred form for high-temperature dideoxy sequencing reactions and for high yield primer extension reactions. The fidelity of polymerization by this form is reduced to a level about 2-fold higher than that of Taq DNA Polymerase (Mattila et al., 1991; Eckert & Kunkel, 1991). Vent_{R} (exo−) DNA Polymerase is an excellent choice for DNA sequencing and is included in our CircumVent Sequencing Kit (see pages 118 and 121).

Both Vent_{R} and Vent_{R} (exo−) are purified from strains of E. coli that carry the Vent DNA Polymerase gene from the archaea Thermococcus litoralis (Perler et al., 1992). The native organism is capable of growth at up to 98° C. and was isolated from a submarine thermal vent (Belkin and Jannasch, 1985). They are useful in primer extension, thermal cycle sequencing and high temperature dideoxy-sequencing.

DEEP VENT_{R}™ DNA Polymerase and DEEP VENT_{R}™ (exo−) DNA Polymerase. Deep Vent_{R} DNA Polymerase is the second high-fidelity thermophilic DNA polymerase available from New England Biolabs. The fidelity of Deep Vent_{R} DNA Polymerase is derived in part from an integral 3′→5′ proofreading exonuclease activity. Deep Vent_{R} is even more stable than Vent_{R} at temperatures of 95 to 100° C. (see graph).

Deep Vent_{R} (exo−) DNA Polymerase has been genetically engineered to eliminate the 3′→5′ proofreading exonuclease activity associated with Deep Vent_{R} DNA Polymerase. This exo− version can be used for DNA sequencing but requires different dNTP/ddNTP ratios than those used with Vent_{R} (exo−) DNA Polymerase. Both Deep Vent_{R} and Deep Vent_{R} (exo−) are purified from a strain of E. coli that carries the Deep Vent_{R} DNA Polymerase gene from Pyrococcus species GB-D (Perler et al., 1996). The native organism was isolated from a submarine thermal vent at 2010 meters (Jannasch et al., 1992) and is able to grow at temperatures as high as 104° C. Both enzymes can be used in primer extension, thermal cycle sequencing and high temperature dideoxy-sequencing.

T7 DNA Polymerase (unmodified). T7 DNA polymerase catalyzes the replication of T7 phage DNA during infection. The protein dimer has two catalytic activities: DNA polymerase activity and strong 3′→5′ exonuclease (Hori et al., 1979; Engler et al., 1983; Nordstrom et al, 1981). The high fidelity and rapid extension rate of the enzyme make it particularly useful in copying long stretches of DNA template.

T7 DNA Polymerase consists of two subunits—T7 gene 5 protein (84 kilodaltons) and E. coli thioredoxin (12 kilodaltons) (Hori et al., 1979; Studier et al., 1990; Grippo & Richardson, 1971; Modrich & Richardson, 1975; Adler & Modrich, 1979). Each protein is cloned and overexpressed in a T7 expression system in E. coli (Studier et al., 1990). It can be used in second strand synthesis in site-directed mutagenesis protocols (Bebenek & Kunkel, 1989).

The reaction buffer is 1× T7 DNA Polymerase Buffer (20 mM Tris-HCl (pH 7.5), 10 mM MgCl₂, 1 mM dithiothreitol). Supplement with 0.05 mg/ml BSA and dNTPs. Incubate at 37° C. The high polymerization rate of the enzyme makes long incubations unnecessary. T7 DNA Polymerase is not suitable for DNA sequencing.

Unit assay conditions are 20 mM Tris-HCl (pH 7.5), 10 mM MgCl₂, 1 mM dithiothreitol, 0.05 mg/ml BSA, 0.15 mM each dNTP, 0.5 mM heat denatured calf thymus DNA and enzyme. Storage conditions are 50 mM KPO₄ (pH 7.0), 0.1 mM EDTA, 1 mM dithiothreitol and 50% glycerol. Store at −20° C.

DNA Polymerase I (E. coli). DNA Polymerase I is a DNA-dependent DNA polymerase with inherent 3′→5′ and 5′→3′ exonuclease activities (Lehman, 1981). The 5′→3′ exonuclease activity removes nucleotides ahead of the growing DNA chain, allowing nick-translation. It is isolated from E. coli CM 5199, a lysogen carrying λpolA transducing phage (obtained from N. E. Murray) (Murray & Kelley, 1979). The phage in this strain was derived from the original polA phage encoding wild-type Polymerase I.

Applications include nick translation of DNA to obtain probes with a high specific activity (Meinkoth and Wahl, 1987) and second strand synthesis of cDNA (Gubler & Hoffmann, 1983; D'Alessio & Gerard, 1988). The reaction buffer is E. coli Polymerase I/Klenow Buffer (10 mM Tris-HCl (pH 7.5), 5 mM MgCl₂, 7.5 mM dithiothreitol). Supplement with dNTPs.

DNase I is not included with this enzyme and must be added for nick translation reactions. Heat inactivation is for 20 min at 75° C. Unit assay conditions are 40 mM KPO₄ (pH 7.5), 6.6 mM MgCl₂, 1 mM 2-mercaptoethanol, 20 μM dAT copolymer, 33 μM dATP and 33 μM ³H-dTTP. Storage conditions are 0.1 M KPO₄ (pH 6.5), 1 mM dithiothreitol, and 50% glycerol. Store at −20° C.

DNA Polymerase I, Large (Klenow) Fragment. Klenow fragment is a proteolytic product of E. coli DNA Polymerase I which retains polymerization and 3′→5′ exonuclease activity, but has lost 5′→3′ exonuclease activity. Klenow retains the polymerization fidelity of the holoenzyme without degrading 5′ termini.

A genetic fusion of the E. coli polA gene, that has its 5′→3′ exonuclease domain genetically replaced by maltose binding protein (MBP). Klenow Fragment is cleaved from the fusion and purified away from MBP. The resulting Klenow fragment has the identical amino and carboxy termini as the conventionally prepared Klenow fragment.

Applications include DNA sequencing by the Sanger dideoxy method (Sanger et al., 1977), fill-in of 3′ recessed ends (Sambrook et al., 1989), second-strand cDNA synthesis, random priming labeling and second strand synthesis in mutagenesis protocols (Gubler, 1987)

Reactions conditions are 1× E. coli Polymerase I/Klenow Buffer (10 mM Tris-HCl (pH 7.5), 5 mM MgCl2, 7.5 mM dithiothreitol). Supplement with dNTPs (not included). Klenow fragment is also 50% active in all four standard NEBuffers when supplemented with dNTPs. Heat inactivated by incubating at 75° C. for 20 min. Fill-in conditions: DNA should be dissolved, at a concentration of 50 μg/ml, in one of the four standard NEBuffers (1×) supplemented with 33 μM each dNTP. Add 1 unit Klenow per μg DNA and incubate 15 min at 25° C. Stop reaction by adding EDTA to 10 mM final concentration and heating at 75° C. for 10 min. Unit assay conditions 40 mM KPO4 (pH 7.5), 6.6 mM MgCl2, 1 mM 2-mercaptoethanol, 20 μM dAT copolymer, 33 μM dATP and 33 μM ³H-dTTP. Storage conditions are 0.1 M KPO₄ (pH 6.5), 1 mM dithiothreitol, and 50% glycerol. Store at −20° C.

Klenow Fragment (3′→5′ exo⁻). Klenow Fragment (3′→5′ exo−) is a proteolytic product of DNA Polymerase I which retains polymerase activity, but has a mutation which abolishes the 3′→5′ exonuclease activity and has lost the 5′→3′ exonuclease (Derbyshire et al., 1988).

A genetic fusion of the E. coli polA gene, that has its 3′→5′ exonuclease domain genetically altered and 5′→3′ exonuclease domain replaced by maltose binding protein (MBP). Klenow Fragment exo− is cleaved from the fusion and purified away from MBP. Applications include random priming labeling, DNA sequence by Sanger dideoxy method (Sanger et al., 1977), second strand cDNA synthesis and second strand synthesis in mutagenesis protocols (Gubler, 1987).

Reaction buffer is 1× E. coli Polymerase I/Klenow Buffer (10 mM Tris-HCl (pH 7.5), 5 mM MgCl₂, 7.5 mM dithiothreitol). Supplement with dNTPs. Klenow Fragment exo− is also 50% active in all four standard NEBuffers when supplemented with dNTPs. Heat inactivated by incubating at 75° C. for 20 min. When using Klenow Fragment (3′→5′ exo−) for sequencing DNA using the dideoxy method of Sanger et al. (1977), an enzyme concentration of 1 unit/5 μl is recommended.

Unit assay conditions are 40 mM KPO₄ (pH 7.5), 6.6 mM MgCl₂, 1 mM 2-mercaptoethanol, 20 μM dAT copolymer, 33 μM dATP and 33 μM ³H-dTTP. Storage conditions are 0.1 M KPO₄ (pH 7.5), 1 mM dithiothreitol, and 50% glycerol. Store at −20° C.

T4 DNA Polymerase. T4 DNA Polymerase catalyzes the synthesis of DNA in the 5′→3′ direction and requires the presence of template and primer. This enzyme has a 3′→5′ exonuclease activity which is much more active than that found in DNA Polymerase I. Unlike E. coli DNA Polymerase I, T4 DNA Polymerase does not have a 5′→3′ exonuclease function.

Purified from a strain of E. coli that carries a T4 DNA Polymerase overproducing plasmid. Applications include removing 3′ overhangs to form blunt ends (Tabor & Struhl, 1989; Sambrook et al., 1989), 5′ overhang fill-in to form blunt ends (Tabor & Struhl, 1989; Sambrook et al., 1989), single strand deletion subcloning (Dale et al., 1985), second strand synthesis in site-directed mutagenesis (Kunkel et al., 1987), and probe labeling using replacement synthesis (Tabor & Struhl, 1989; Sambrook et al., 1989).

The reaction buffer is 1× T4 DNA Polymerase Buffer (50 mM NaCl, 10 mM Tris-HCl, 10 mM MgCl₂, 1 mM dithiothreitol (pH 7.9 at 25° C.)). Supplement with 40 μg/ml BSA and dNTPs (not included in supplied 10× buffer). Incubate at temperature suggested for specific protocol.

It is recommended to use 100 μM of each dNTP, 1-3units polymerase/μg DNA and incubation at 12° C. for 20 min in the above reaction buffer (Tabor & Struhl, 1989; Sambrook et al., 1989). Heat inactivated by incubating at 75° C. for 10 min. T4 DNA Polymerase is active in all four standard NEBuffers when supplemented with dNTPs.

Unit assay conditions are 50 mM NaCl, 10 mM Tris-HCl, 10 mM MgCl₂, 1 mM dithiothreitol (pH 7.9 at 25° C.), 33 μM dATP, dCTP and dGTP, 33 μM ³H dTTP, 70 μg/ml denatured calf thymus DNA, and 170 μg/ml BSA. Note: These are not suggested reaction conditions; refer to Reaction Buffer. Storage conditions are 100 mM KPO₄ (pH 6.5), 10 mM 2-mercaptoethanol and 50% glycerol. Store at −20° C.

I. Kits

All the essential materials and reagents required for performing cDNA preparation, dsRNA production, cell culturing and various assays may be assembled together in a kit. Such kits generally may comprise comprise polymerases (reverse transcriptases, DNA polymerases), restriction enzymes, ligase, dNTPs, buffers to provide the necessary reaction mixture for DNA and RNA synthesis, primers, all of which are described above. All of the kits will provide suitable container means for storing and dispensing these reagents.

### J. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

General cell culture methods. Embryos are isolated by treating adult nematodes with an alkaline hypochlorite solution (0.5 M NaOH and 1% NaOCl) for 5 min (Edgar, 1995). Eggs released by this treatment are pelleted by centrifugation and then washed 3× with egg buffer containing 118 mM NaCl, 48 mM KCl, 2 mM CaCl₂, 2 mM MgCl₂ and 25 mM Hepes (pH 7.3, 340 mOsm). Adult carcasses are separated from washed eggs by centrifugation in 30% sucrose. The egg layer is removed by pipette and washed once with egg buffer and then pelleted. Eggshells are removed by re-suspending pelleted eggs for 20-30 min in egg buffer containing 1 U/ml of chitinase (Sigma Chemical Co., St. Louis, Mo.).

After eggshell removal, embryo cells are dissociated by gentle pipetting. Older embryos that have undergone significant morphogenesis are not dissociated by this treatment. Dissociated cells are filtered through a 10 μm Duropore filter to remove intact embryos and newly hatched larvae. Filtered cells are plated onto glass coverslips or plastic cell culture dishes in L15 medium containing 10% fetal bovine serum. Medium osmolality is adjusted to 340 mOsm with sterile sucrose. All culture surfaces are pre-treated with peanut lectin to promote cell adherence and differentiation (Buechner et al., 1999). Cultures are at maintained at 20-23° C. in a humidified incubator equilibrated with room air.

For ongoing experiments, the inventors typically harvest 2-3 ml of gravid adult worms from 4-6 ten cm culture plates. After alkaline hypochlorite treatment, approximately 100-200 μl of eggs are recovered. Chitinase treatment usually yields 20-50 million cells. These cells are seeded at a density of 300,000-375,000 cells/cm².

The cell cultures currently generated are well-suited for patch clamp and imaging studies. In order to purify or partially purify various cell types, it will be necessary to scale up our worm cultures. To accomplish this goal, worms will be synchronized by growth in liquid culture until the culture “starves” and larvae enter the dauer state (Lewis & Fleming, 1995). These animals will be plated on multiple 15 cm culture plates seeded with E. coli and grown until they are gravid. Gravid adults will be harvested for blastomere isolation.

FACS methods. Cells are sorted using a FACStar Plus (Becton Dickinson) equipped with a 488 nm argon laser and FITC filter set (emission: 530/60 BP) for sorting GFP-expressing cells. Methods for sorting GFP-labeled cells are well-established.

Two general FACS protocols are used. For cell types in which GFP reporters are expressed in blastomeres, FACS is performed immediately after cell isolation. In the case of cells that require differentiation before GFP reporters are expressed, blastomeres are isolated and cultured for 1-2 days. Cultured cells are dissociated from the growth surface by brief treatment with Ca²⁺-free medium or trypsin.

After sorting and plating cells, detailed light microscopy studies are performed in order to assess purity and both short- and long-term cell viability. The purity of sorted cells is quantified by counting the total number of cells using DAPI staining and determining the percent of these cells expressing GFP. Measurement of purity is made immediately after plated cells have adhered to the growth substrate. GFP expression will be monitored daily for 3 days after cell plating.

Cell viability is assessed using a commercially available LIVE/DEAD Viability/Cytotoxicity kit (Molecular Probes). The assay is rapid and relies on the use of membrane-permeant calcein AM and membrane-impermeant ethidium homodimer-1. Calcein AM permeates lives cells and is cleaved by endogenous esterases producing green fluorescence. Ethidium permeates only dead cells and stains their nucleic acids with red fluorescence. LIVE/DEAD assays are performed using conventional longpass fluorescein filter sets.

The evidence obtained indicates that early embryonic blastomeres are present in the population of freshly isolated embryonic cells that differentiate in culture. The embryonic lineage predicts that many of these cells should be programmed to undergo several rounds of division in vitro. The inventors will characterize the mitotic potential of cultured cells in two ways. First, cultured cells will be assayed for incorporation of BromoDeoxyUridine (BrDU). The presence of this nucleotide homolog in nascent DNA can be detected by staining with a BrDU-specific antibody. BrDU will be added to the culture media for a 2 h pulse and then removed by washing. Fixation and staining methods will be the same as those described by Boxem et al. (1999). BrDU-treated cells will be counterstained with the DNA specific dye, DAPI, and visualized by fluorescence microscopy. The fraction of cells undergoing mitosis and DNA synthesis will be measured by comparing BrDU stained cells with the total population of DAPI-stained nuclei.

The inventors also will assess the mitotic potential of cultured cells by direct observation with a fully motorized Zeiss IM200 inverted microscope that can be used in a time lapse imaging mode to track cell divisions. The expression of cell-specific GFP markers in these experiments should allow addressing of the intriguing question of whether these embryonic cells are capable of recapitulating the stereotypical patterns of cell divisions that they undergo in intact embryos.

GFP reporters to be used in sorting experiments. Cell-specific GFP reporters have revealed that C. elegans cells in culture differentiate into muscle cells and specific classes of neurons. The inventors have now obtained additional GFP transgenic worm strains that will allow us to detect differentiation of a variety of other C. elegans cell types. The following cell types will be used in FACS methods with various GFP reporters.

Hypodermal cells. The worm “skin” or hypodermis is an epithelium that underlies the cuticle. Hypodermal cells arise from the AB and C founder cells and eventually envelop the embryo. Dorsally located hypodermal cells fuse to create a large multinucleated synctial cell called hyp 7. The hypodermis secretes the cuticle and functions as a storage site for lipid droplets (White, 1988). Hypodermal cells also function as neuroglia and secrete a basal lamina that provides a substrate for migrating mesodermal cells and neuronal growth cones (Hedgecock et al., 1990; Wadsworth & Hedgecock, 1992). Recent studies on the ClC anion channel homolog, CLH-1, suggest that the hypodermis may also have an osmoregulatory function (Petalcorin et al., 1999).

lin-26 encodes a zinc-finger protein necessary for differentiation of non-neuronal ectodermal cells. LIN-26 expression is initially detected in embryonic hypodermal cells soon after their birth and persists in all hypodermal cells into the adult stage (Labouesse et al., 1994; 1996).

Seam cells. The inventors also have obtained a GFP reporter that is exclusively expressed in seam cells (“seam-cell”::GFP), a linear array of hypodermal cells that do not fuse with dorsal hyp7 cells. Differentiation of seam cells requires the expression of a unique combination of genes suggesting that these cells carry out specialized functions in the hypodermis (Terns et al., 1997). One of these functions is secretion of a specialized cuticle that forms the lateral alae (Singh & Sulston, 1978).

Intestinal cells. The nematode intestine is comprised of a single layer of 20 epithelial cells. These cells play a critical role in nutrient absorption and yolk protein production. Intestinal cells have also been shown to protect the animal from substances such as heavy metals and toxins (Koga et al., 2000; Lincke et al., 1993) and may play a role in whole animal ionic and osmotic homeostasis.

All of the intestinal cells arise from a single embryonic founder cell, the E blastomere. E blastomeres placed in culture differentiate into epithelial cells that produce apical tight junctions, express proteins exhibiting a polarized distribution, and surround an extracellular space analogous to the intestine lumen (Leung et al., 1999).

The inventors will isolate intestinal precursor cells from a transgenic worm strain line expressing a gut-specific reporter, elt-2::GFP. elt-2 encodes a GATA factor required for formation of the intestine (Fukushige et al., 1998). elt-2::GFP is expressed in E blastomere daughter cells beginning at the 28 cell stage in early embryos. Gut-specific expression persists in embryos, larvae and adults.

Excretory cells. The worm “kidney” is comprised of three cells types, the excretory cell, the duct cell and the pore cell (Nelson et al., 1983). Destruction of any of these cells by laser ablation causes the animals to swell with fluid and die (Nelson & Riddle, 1984).

The excretory cell is a large, H-shaped cell that sends out processes both anteriorly and posteriorly from the cell body. A fluid-filled excretory canal is surrounded by the cell cytoplasm. The basal cell pole of the cell faces the pseudocoel while the apical membrane faces the excretory canal lumen. Gap junctions connect the excretory cell to the hypodermis suggesting an interaction between the two cell types that may be important for whole animal osmoregulation and/or excretion of waste products.

An excretory duct connects the excretory canal to the outside surface of the worm. The duct is formed from cuticle that is continuous with the animal's exoskeleton. The upper two-thirds of the duct is surrounded by the duct cell. A pore cell surrounds the lower third of the duct. The excretory cell is a single-cell “epithelium” that appears to secrete salt, water and waste products into the excretory canal. The duct cell may also play an important role in solute and water transport. Apical surface area of the duct cell is greatly amplified by extensive invaginations and the cytoplasm is filled with mitochondria. Nelson et al. (1983) have suggested that the duct cell may be involved in selective solute reabsorption. If this is the case, the nematode excretory and duct cells are analogous to the acini and ducts of mammalian secretory epithelia such as the salivary gland, sweat glands and pancreas.

Excretory cells have been observed in the inventors' cultures, albeit at a very low frequency. The morphology of these cells is similar to that described previously by Buechner et al. (1999). Excretory cells in vitro have one or two well-developed processes that presumably represent the excretory canals that run the length of the animal's body. A highly refractile region runs through the cell body into the processes. This region is the canal lumen.

Neurons and body wall muscle cells. Newly hatched L1 larvae have 81 body wall muscle cells and 222 neurons. As discussed earlier, these cells represent the majority of cells present in culture. GFP driven by the myo-3 promoter is an excellent reporter for body wall muscle cells. Numerous GFP reporters are available for various types of neurons.

Pharyngeal muscles. The nematode pharynx is a muscular pump that functions to ingest and grind food. Pharyngeal muscle contraction has been studied extensively and the pharynx has proven to be a valuable model for defining the molecular/genetic basis of excitable cell function (e.g., Davis et al., 1999). The electrical excitability of the pharynx is intrinsic to pharyngeal muscle cells. Pharyngeal action potentials resemble those of ventricular cells in the vertebrate heart. ceh-22 encodes an NK-2 class homeodomain transcription factor that is expressed exclusively in pharyngeal muscle cells (Okkema et al., 1997). The CEH-22 homeodomain is most similar to Drosophila tinman and vertebrate homologs that are specifically expressed in the developing heart. ceh-22::GFP expression is initiated midway through embryonic development (the “bean” stage) in most of pharyngeal muscle cells and continues into the adult. The pharyngeal identity of these cells in culture will be confirmed by staining with antibodies that are specific to the pharyngeal myosins MYO-1 and MYO-2 (Miller et al., 1986).

Enteric muscles. Four specialized muscle cells are attached to the posterior end of the intestine where they regulate the opening of the anal pore and expulsion of intestinal contents. A pair of intestinal muscles contract coordinately with the anal sphincter and anal depressor muscles during expulsion. GABA functions as an excitatory neurotransmitter for these muscles which distinguishes them physiologically from the body wall striated muscles for which GABA is inhibitory. arg-1 encodes an “apx-1 related gene” and is believed to function as a NOTCH/LIN-12 activating ligand. The inventors obtained an arg-1::GFP transgenic line that is expressed in the enteric muscles and in the Head Mesodermal Cell (HMC), a large cell of unknown function adjacent to the posterior bulb of the pharynx. The enteric muscles also express the MYO-3 and UNC-54 myosins, which can be detected by staining with specific antibodies (Ardizzi and Epstein, 1987).

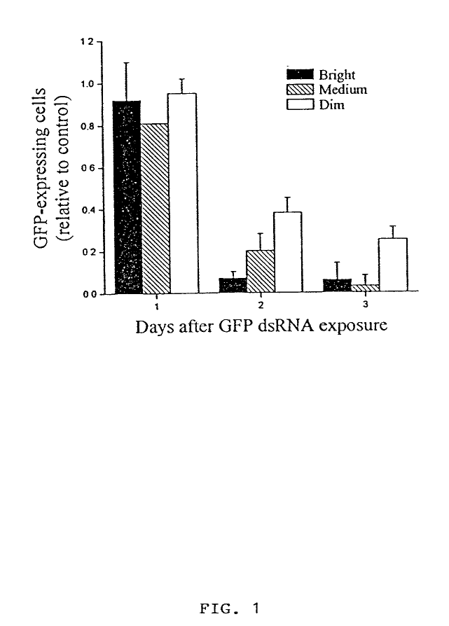

dsRNA treatment protocol for disrupting gene expression. The RNA₁ results shown in FIG. 1 were produced by exposing cells to 10 μg dsRNA/ml. While knockdown of GFP is substantial, it is not complete. Therefore, to further characterize the effectiveness of dsRNA in culture, detailed dose-response studies will be conducted. Both the extent and time course of knockdown of gene expression will be determined. unc-54 expression in muscle cells will also be measured by western analysis.

For GFP RNA₁ studies, cells are grown in multi-well chamber slides with glass cover slip bottoms. Cultures are imaged daily for five days after exposure to dsRNA. Control and dsRNA-treated wells will be seeded identically. Random fields are imaged in control and experimental wells. The number of GFP-positive cells in each field are quantified relative to the total number of cells. In addition, mean pixel fluorescence intensity are quantified in GFP-positive cells using MetaMorph software.

unc-54 dsRNA studies are performed by growing cells in single well chamber slides seeded at equal densities. Total protein is extracted from control and dsRNA-treated cultures wells daily for five days after exposure to dsRNA. Western analysis is performed using standard methods. Protein content in cell extracts is determined by protein assay and gel lanes will be loaded with equal total protein.

Double-stranded RNA (dsRNA) was synthesized using established methods (Fire et al., 1998). Briefly, a DNA template encoding nucleotides 5236-5851 of unc-54 mRNA was obtained by RT-PCR (Miller and Niemeyer, 1995). The vector pPD79.44 was used for GFP dsRNA synthesis. Sense and antisense RNA were synthesized by T3 and T7 polymerase reactions (MEGAscript kit, Ambion, Austin, Tex.). Template DNA was digested with DNaseI and RNA purified by ethanol precipitation. dsRNA was formed by dissolving purified RNA in RNase-free water and then heating to 65 C. for 30 min followed by cooling to room temperature. The size, purity, and integrity of dsRNA were assayed on TAE agarose gels.

Equal numbers of cells from a cell isolate were plated in either control L-15 cell culture medium or L-15 medium containing 15 μg/ml dsRNA. Two to three hours after plating, the dsRNA was diluted to a final concentration of 5 μg/ml. Gene expression was quantified in parallel in both control and dsRNA-treated cells and is expressed relative to that observed in the control cultures.

Electrophysiology. Cells are patch clamped using either the conventional whole-cell mode or perforated patch method. Initially, whole-cell measurements are performed using “physiological” pipette and bath solutions. The composition of C. elegans extracellular and intracellular fluids is currently unknown. Ascaris bath Ringer's (Richmond & Jorgenson, 1999) and conventional high K⁺, low Na⁺ and Cl⁻ pipette solutions are used. Standard voltage clamp protocols are used to assess the voltage-dependence of channels responsible for whole-cell currents.

“Physiological” whole-cell current measurements are followed by standard experiments designed to isolate and characterize specific anion and cation currents. For example, Cl⁻ currents are studied in isolation by using NMDG-Cl bath and pipette solutions. Specific current types are characterized further by performing a limited series of pharmacological inhibitor studies. The intracellular Ca²⁺- and ATP-dependence of all currents observed will be assessed.

Excretory cell fluid transport. As discussed earlier, excretory cells are present, albeit at low frequency, in the inventors' mixed cultures. The appearance of the cells is similar to that described by Buechner et al. (1999). They typically have one or two well-developed processes. A single, highly refractile canal-like structure extends from the tip of the processes across the cell body. This structure is the excretory canal.

A number of observations indicate that the excretory cell is a secretory cell responsible for elimination of fluid and waste products from the pseudocoelomic space. For example, exposure of worms to hypotonic media causes increased fluid secretion and excretion (Nelson & Riddle, 1984). Animals swell with fluid and die when excretory cells are laser ablated (Nelson & Riddle, 1984). Mutations in various genes leads to the formation of large, fluid-filled cysts in excretory cell tubular processes (Buechner et al., 1999).

Fluid secretory activity of cultured excretory cells is tested using quantitative video microscopy. Fluid secretion are assessed by quantifying changes in the volume of the excretory canal. Cells are imaged at 100× by video-enhanced DIC microscopy. Images are recorded on videotape or computer hard drive and analyzed off-line using MetaMorph or Optimas image analysis software. Changes in canal volume are monitored by measuring canal length and width at a single focal plane. Imaging methods used to characterize salt and water transport in epithelial cells, as well as a variety of other cell types, have been reported (Churchwell et al., 1996; Strange & Spring, 1986; 1987a 1987b).

### Example 2

### Results

Differentiation of blastomeres in culture. Freshly isolated blastomeres plated onto peanut lectin-treated plastic petri dishes or glass cover slips undergo striking differentiation. Within 2-3 h after plating, cells can be observed sending out neurite-like processes. Differentiation continues for at least 24-48 h. The majority of cells in culture have neuronal-like morphology or a spindle shape typical of striated muscles. Other differentiated cell types including excretory cells and epithelial-like cells are also observed.

It should be noted and stressed here that treatment of the culture chamber with lectin or other agents that allow cells to adhere to the growth substrate is essential for differentiation. In their elegant study of excretory cell differentiation, Buechner et al. (1999) noted that blastomeres differentiate in vitro when plated onto peanut lectin-coated glass cover slips. The present inventors have observed that blastomeres remain viable in culture for at least 1 week when plated in the absence of peanut lectin. However, the vast majority of cells fail to undergo any obvious morphological differentiation.

The high proportion of neurons and muscle cells in culture reflects their relative abundance in vivo. Adult hermaphrodites and newly hatched L1 larvae are comprised of 959 and 550 cells, respectively. Neurons and muscles thus represent ˜40% of the adult cells and ˜55% of cells in the L1 larva.

A powerful experimental advantage of C. elegans is the relative ease and economy of generating transgenic animals. It is a mainstay in the field to use GFP reporters for cellular localization of gene expression (Chalfie et al., 1994). To further examine cell differentiation, the inventors cultured cells from various GFP reporter worm strains. In all cases, the inventors observed striking expression of GFP reporters in cultured cells. The relative proportion of cells expressing a particular reporter was similar to that observed in the intact animal. For example, most, if not all, neurons in vivo express unc-119 (Maduro & Pilgrim, 1995). A newly hatched L1 larva is comprised of 550 cells, 222 or 40.4% of which are neurons. Approximately 45% of cells in culture express unc-119::GFP. Virtually all GFP-positive cells have a neuron-like morphology. GFP-positive cells are also present in freshly dissociated blastomeres, which is consistent with the observation that unc-119::GFP is expressed in neuronal precursors in vivo beginning at the ˜60 cell stage (Maduro & Pilgrim, personal communication). unc-4 encodes a homeodomain transcription factor (Miller et al., 1992) that is expressed in the 13 embryonic cholinergic motor neurons, which represent 2.4% of the 550 cells that comprise the newly hatched L1 larva. GFP-positive cells are present at a frequency of 2-4% in cultures produced from unc-4::GFP transgenic worms.

unc-4::GFP-expressing neurons first appear about midway through embryonic development (˜400 min) after morphogensis has begun (Miller et al., 1995). Freshly prepared blastomeres from unc-4::GFP embryos rarely show GFP expression. This finding is consistent with our observation that cells from late stage embryos are not present in our isolated cell preparation. Expression of unc-4::GFP is detected within 12-24 h after plating cells.

Interestingly, the inventors have observed unc-4-expressing neurons sending out processes that made physical contact with spindle shaped muscle cells. Cholinergic motor neurons form neuromuscular junctions with striated body wall muscles in vivo. A specific synaptic vesicle protein, synaptotagmin (SNT-1) that functions at these neuromuscular synapses (Lickteig et al., 2001) also is expressed by unc-4::GFP motor neurons in vitro. Taken together, these observations indicate that unc-4::GFP-expressing cells recapitulate functional properties observed in vivo.

myo-3 and unc-54 encode specific myosin heavy chain isoforms that are co-expressed in body muscles but are not detected in pharyngeal muscles (Ardizzi & Epstein, 1987; Miller et al., 1983). The myo-3 reporter worm strain expresses two GFPs with peptide signals that target them to either the nucleus or mitochondria. Muscle cells in culture show GFP expression in both the nucleus and in elongated intracellular structures that are likely to be mitochondria. All myo3::GFP-positive muscle cells in culture also show immunofluorescence localization of UNC-54 myosin.

An important question is whether cells in culture are exhibiting embryonic or postembryonic differentiation. To begin addressing this issue, the inventors cultured cells from worm strains expressing del-1::GFP. del-1 encodes a DEG/ENaC-like channel that is expressed in 2 embryonic neurons (SABVL and SABVR). In L2 and L3 larvae, del-1 is expressed in 11 VB neurons and 12 VA neurons, respectively (Winnier et al., 1999). Thus, if del-1-expressing neurons undergo post-embryonic development in vitro, the frequency of del-1::GFP reporter expression should be relatively high compared to that observed in the intact embryo.

The total number of del-1::GFP-labeled cells in random fields of cells cultured in chamber slides with glass cover slip bottoms were counted. The frequency of del-1 expression should be 2 out of every 550 cells or ca. 0.36% if the cultures are recapitulating embryonic differentiation. This predicted value is remarkably close to the observed frequencies of 0.33-0.46%. If post-embryonic differentiation were occurring, expression frequency should be considerably higher.

The conclusion that post-embryonic VA and VB motor neurons are not differentiating in culture also is consistent with an experiment conducted with del-1::GFP in an unc-4 mutant background. In an unc-4 null mutant, del-1::GFP expression is no longer detected in the embryonically derived SAB neurons (Miller, unpublished observations), but is retained in the VA and VB motor neurons (Winnier et al., 1999). del-1::GFP expression was not detected in cell cultures derived from unc-4 mutant worms.

Taken together, these experiments suggest that the blastomeres cultured in vitro do not undergo post-embryonic differentiation. However, it is important to further characterize the spectrum of cell types that differentiate in culture. An important goal of the current proposal is to extend our studies to the expression of cell-specific markers for various epithelial cell types and additional classes of neurons and muscle cells.

Survival of blastomeres in culture. The inventors have not as yet attempted to rigorously quantify cell survival. However, daily measurements of GFP expression in single cultures for up to 6 days after plating have been performed and have seen no obvious decrease in cell survival. In addition, differentiated cells have been patch clamped up to 2 weeks after blastomere isolation with no difficulty. An important goal of this proposal is to quantify long-term cell viability.

Reverse genetic screening of cultured cells: in vitro RNA-mediated gene interference. RNAᵢ is a powerful tool for disrupting gene expression in vivo. RNA₁ has also been demonstrated to work effectively in Drosophila S2 cell lines (Caplen et al., 2000; Clemens et al., 2000; Ui-Tei et al., 2000). Early cell culture attempts were motivated by a desire to use RNAᵢ in vitro in an effort to identify genes encoding novel anion channels. To test the effectiveness of RNAᵢ in culture, the inventors attempted to disrupt the expression of GFP driven by the unc-4 promoter in cholinergic motor neurons. This experiment posed two important challenges. First, GFP is encoded by a transgene and is therefore typically overexpressed compared to endogenous cellular genes. Compared to native proteins, knockdown of GFP levels is expected to be somewhat more difficult.

A second and particularly important point concerns the effectiveness of RNA₁ in neurons. It is generally accepted that “systemic” exposure of C. elegans to dsRNA has little effect on neuronal gene expression (Tavernarakis et al., 2000; Timmons et al., 2001). However, neurons can be engineered to express dsRNA transcribed from inverted repeat transgenes (Tavernarakis et al., 2000). The so-called “snapback” dsRNA constructs are effective in disrupting neuronal gene expression indicating that neurons possess the molecular machinery required for RNAᵢ. The lack of an RNAᵢ effect during systemic dsRNA exposure suggests that dsRNA does not readily cross the neuronal membrane in vivo.

FIG. 1 shows the effect of GFP dsRNA on GFP levels in cholinergic motor neurons. Three days after addition of dsRNA to the culture, GFP expression was reduced >80-95%. These results indicate that C. elegans neurons in culture are readily susceptible to exogenous RNAᵢ.

Assay of cultured cell functional properties. As discussed earlier, access to nematode somatic cells for direct physiological measurements of functional properties is, at best, technically demanding. The ability to perform electrophysiological and quantitative imaging studies on cultured cells would provide important new opportunities for the molecular characterization of membrane transport processes. The inventors have begun to assess the “patch clampability” of cultured neurons and muscle cells. While the cells are generally smaller than mammalian cells, they have found that they can be patch clamped readily in the conventional whole-cell and isolated patch modes.

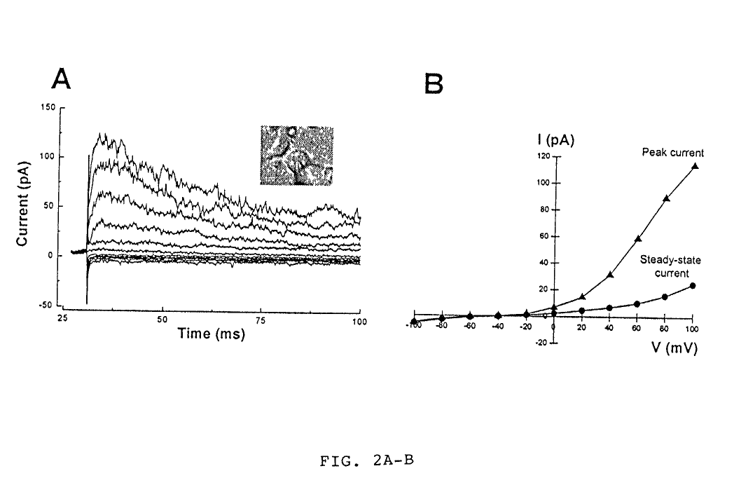

FIG. 2 illustrates a typical whole-cell recording from a cultured C. elegans neuron. The inventors routinely observe a strongly outwardly rectifying current exhibiting time-dependent inactivation when cells are patch clamped with a high K⁺ pipette solution. These currents are remarkably similar to K⁺ currents detected in C. elegans ASER neurons by Goodman et al (1998).

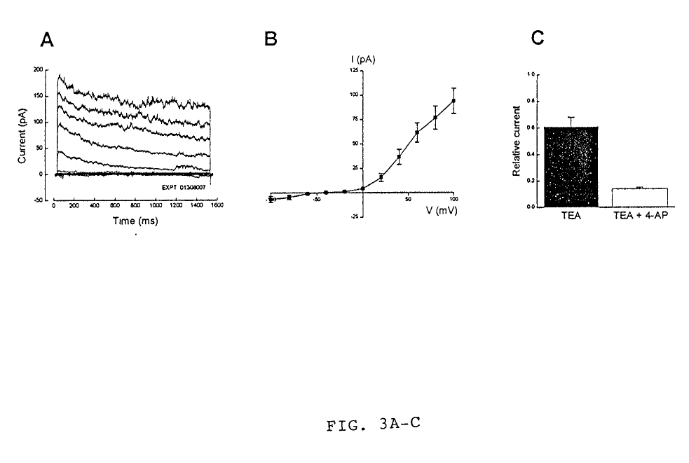

FIG. 3 shows whole-cell recordings from myo-3::GFP-expressing muscle cells. For these experiments, the inventors mimicked the experimental protocol and bath and pipette solutions used recently for in vivo patch clamp studies of C. elegans body wall muscles (Richmond et al., 1999). When cultured body muscles were patch clamped with a pipette solution containing 120 mM KCl (Richmond et al., 1999), slowly inactivating, strongly outwardly rectifying currents were observed (FIG. 3). These currents were inhibited 40% by 20 mM TEA and 85% by 20 mM TEA plus 3 mM 4-aminopyridine (4-AP). The results shown in FIG. 3 are remarkably similar to those reported by Richmond and Jorgensen using the so-called “filleted worm” preparation (Richmond et al., 1999). The similarity between in vivo and in vitro patch clamp recordings argues that primary cultures of body muscles recapitulate at least some of their native functional properties.

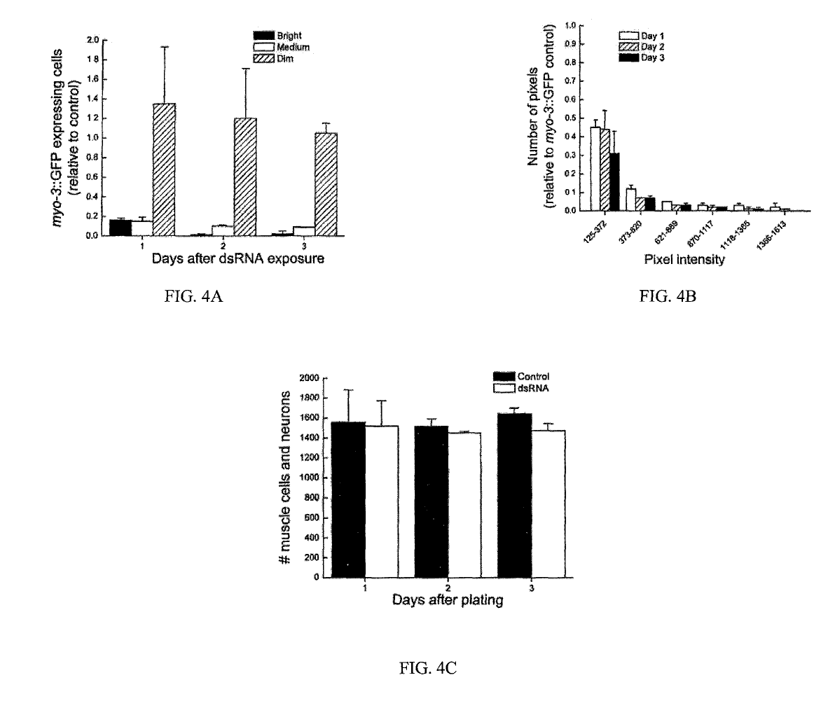

Double-Stranded RNA Disrupts Targeted Gene Expression in Cultured Embryonic Cells. A typical fluorescence micrograph of control myo-3::GFP-expressing cells and cells treated with GFP dsRNA for 3 days was taken. GFP levels were quantified by scoring cells as expressing bright, medium, or dim GFP fluorescence. As shown in FIG. 4A, the number of cells exhibiting bright and medium GFP fluorescence was reduced by 85% -90% (FIGS. 4A and 4B) within 24 hr of treatment with dsRNA.

GFP levels were also quantified by measuring the intensity of all pixels in each fluorescence image. As shown in FIG. 4B, there was a 50% to >90% reduction in the number of pixels within the measured intensity ranges in images of cells treated with dsRNA. This effect was maximal within 24 hr after dsRNA exposure.

To ensure that the reduction in GFP fluorescence was not due to a loss of cells in the dsRNA-treated cultures, the total number of cells in each field that had muscle and neuronal morphology were also counted. As shown in FIG. 4C, the number of muscle cells and neurons in both control and dsRNA-treated cultures was similar.

The effect of dsRNA on the expression of the native, myosin-encoding gene unc-54 (Epstein et al., 1974; Miller et al., 1983) was also examined. UNC-54 is virtually undetectable in the dsRNA-treated cells. The mean dsRNA-induced reduction of UNC-54 expression was 94±3% (mean±SE; n=3).

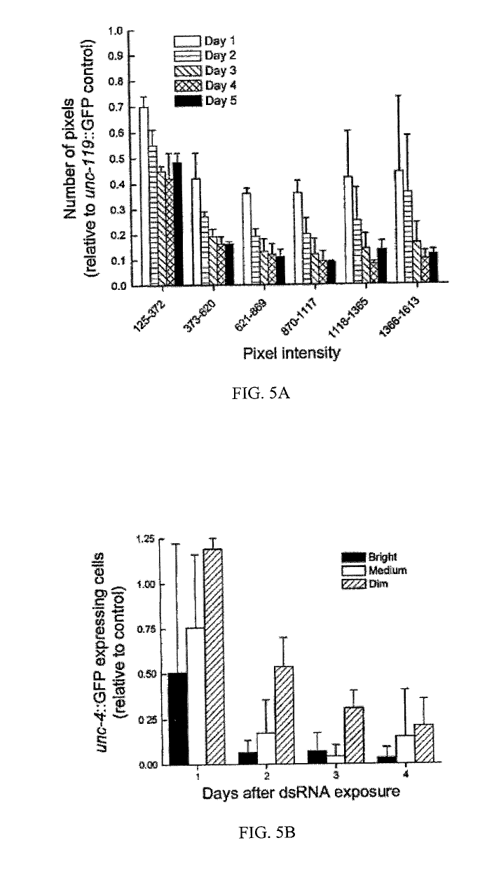

To assess the effectiveness of RNAi on neurons in vitro, GFP expression in cultures derived from the unc-119::GFP worms was monitored. As shown in FIG. 5A, GFP dsRNA dramatically reduced GFP expression in cultured neurons. However, unlike myo-3::GFP, downregulation of unc-119::GFP was considerably slower. Knockdown of GFP expression in muscle cells was largely complete one day after exposure to dsRNA. In contrast, reduction of unc-119::GFP appeared to be maximal 4 days after dsRNA treatment (FIG. 5A). The slower reduction of GFP expression in dsRNA-treated cultured neurons is consistent with in vivo observations (Timmons et al., 2001).

The effectiveness of dsRNA on neuronal gene expression was examined further by monitoring GFP fluorescence in cells cultured from unc-4::GFP transgenic worms. unc-4-expressing cells were scored manually as expressing bright, medium or dim GFP fluorescence. As shown in FIG. 5B, GFP fluorescence levels were only modestly affected 1 day after dsRNA exposure. However, 3 days after treatment with dsRNA, there was a >90% reduction in the number of cells expressing bright and medium GFP fluorescence levels.

### K. REFERENCES

The following references, to the extent that they provide exemplary procedural or other details supplementary to those set forth herein, are specifically incorporated herein by reference.

U.S. Pat. No. 4,704,362

U.S. Pat. No. 5,221,619

U.S. Pat. No. 5,583,013

U.S. Pat. No. 4,659,774

U.S. Pat. No. 4,816,571

U.S. Pat. No. 4,959,463

U.S. Pat. No. 5,141,813

U.S. Pat. No. 5,264,566

U.S. Pat. No. 5,428,148

U.S. Pat. No. 5,554,744

U.S. Pat. No. 5,574,146

U.S. Pat. No. 5,602,244

Alberts et al., In: Molecular Biology of the Cell, Robertson (Ed.), Garland, New York, p 369, 1994.

Ardizzii and Epstein, “Immunochemical localization of myosin heavy chain isoformis and paramyosin in developmentally and structurally diverse muscle cell types of the menatode Caenorhabditis elegans,” J. Cell. Biol., 105:2763-2770, 1987.

Beaucage, and Lyer, Tetrahedron, 48:2223-2311, 1992.

Bebenek & Kunkel, Nucl. Acids Res., 17:5408, 1989.

Belkin & Jannasch, Arch. Microbiol., 141:181-186, 1985.

Boguski, Trends Biochem. Sci., 21:295-296, 1995.

Bosher and Labouesse, “RNA interference; genetic wand and genetic watchdog,” Nat. Cell Biol., 2:E31-E36, 2000.

Boxem, Srinivasan, D. H. Van, “The Caenorhabditis elegans gene ncc-1 encodes a cdc2-related kinase required for M phase in meiotic and mitotic cell divsions, but not for S phase,” Development, 126:2227-2239, 1999.

Buechner et al., Dev. Biol. 214:227-241, 1999.

Caplen, N. J., Fleenor, J., Fire, A., and Morgan, R. A. “dsRNA mediated gene silencing in cultured Drosophila cells: a tissue culture model for the analysis of RNA interference,” Gene 252, 95-105, 2000.

Chalfie, Tu, Euskirchen, Ward, Prasher, “Green fluorescent protein as a marker for gene expression,” Science, 263:802-805, 1994.

Churchwell, Wright, Emma, Rosenberg, Strange, “NMDA receptor activation inhibits neuronal volume regulation following swelling induced by veratridine-stimulated Na⁺ influx in rat coritcal cultures,” J. Neurosci., 16:7447-7457, 1996.

Clemens, J. C., Worby, C. A., Simonson-Leff, N., Muda, M., Maehama, T., Hemmings, B. A., and Dixon, J. E. “Use of double-stranded RNA interference in Drosophila cell lines to dissect signal transduction pathways,” Proc. Natl. Acad. Sci. USA 97, 6499-6503, 2000.

Cowley, Jr., J. Vasc. Res., 36:83-90, 1999.

Dale et al., Plasmid, 13:31-40, 1985.

D'Alessio and Gerard, Nucl. Acids Res., 16:1999-2014, 1988.

Davis, Fleischhauer, Dent, Joho, Avery, “A mutation in the Caenorhabditis elegans EXP-2 potassium channel that alters feeding behavior,” Science, 286:2501-2504, 1999.

Derbyshire et al., Science, 240:199-201, 1988.

Eckert & Kunkel, PCR Methods and Applications, 1:17-24, 1991.

Edgar, “Blastomere culture and analysis,” Methods Cell Biol., 48:303-321, 1995.

Engler et al., J. Biol. Chem., 258:11165-11173, 1983.

Epstein, H. F., Waterston, R. H., and Brenner, S. “A mutant affecting the heavy chain of myosin in Caenorhabditis elegans,” J. Mol. Biol. 90, 291-300, 1974.

Fire et al., Nature, 391:806-811, 1998.

Fukushige, Hawkins, McGhee, “The GATA-factor elt-2 is essential for formation of the Caenorhabditis elegans intestine,” Dev. Biol., 198:286-302, 1998.

Gerhold & Caskey, BioEssays, 18:973-981, 1996.

Gillam et al., J. Biol. Chem., 253:2532, 1978.

Gillam et al., Nucleic Acids Res., 6:2973, 1979.

Goodman , Hall, Avery, Lockery, “Active currents regulate sensitivity and dynamic range in Caenorhabditis elegans neurons,” Neuron, 20:763-772, 1998.

Grippo & Richardson, J. Biol. Chem., 246:6867-6873, 1971.

Grishok et al., Science, 287:2494-2497, 2000.

Gubler & Hoffmann, Gene, 25:263-269, 1983.

Gubler, Methods Enzymol., 152:330-335, 1987.

Hedgecock, Culotti, Hall, “The unc-5, unc-6 and unc-40 genes guide circumferential migrations of pioneer axons and mesodermal cells on the epidermis in Caenorhabditis elegans,” Neuron, 4:61-85, 1990.

Hodgkin et al., Science, 270:410-414, 1995.

Hori et al., J. Biol. Chem., 254:11598-11604, 1979.

Houts et al., J. Virol., 29:517-522, 1979.

Hugh and Griffin, PCR Technology, 228-229, 1994.

Iiyy et al., Biotechnique, 11:464, 1991.

Itakura and Riggs, Science, 209:1401-1405, 1980.

Itakura et al., J. Biol. Chem., 250:4592 1975.

Jannasch et al., Applied Environ. Microbiol., 58:3472-3481, 1992.

Kao, Biotechnol. Prog., 15:304-311, 1999.

Ketting et al., Cell, 99:133-141, 1999.

Khorana, Science, 203, 614 1979.

Koga, Zwaal, Guan, Avery, Ohshima, “A Caenorhabditis elegans MAP kinase kinase, MEK-1, is involved in stress responses,” EMBO J., 19:5148-5156, 2000.

Kong et al., J. Biol. Chem., 268:1965-1975, 1993.

Kunkel et al., Methods Enzymol., 154:367-382, 1987.

Labouesse, Hartweig, Horvitz, The Caenorhabditis elegans LIN-26 protein is required to specify the factes of jypodernal cells and encodes a presumptive zinc-finger transcription factor,“Development, 120:2359-2368, 1994.

Lehman, In: The Enzymes, Boyer (Ed.), Vol. 14A, pp 16-38, Academic Press, San Diego, Calif., 1981.

Leung, Hermann, Priess, “Organogenesis of the Caenorhabditis elegans intestine,” Dev. Biol., 216:114-134, 1999.

Lewis and Fleming, “Basic culture methods,” In: Caenorhabditis elegans: Modern Biological Analysis of an Organism, Epstein and Shakes (Eds.), Academic Press, New York, 4-29, 1995.

Lickteig, Duerr, Frisby, Hall, Rand, Miller, “Regulation of neurotransmitter vesicles by the homeodomain protein UNC-4 and its transcriptional corepressor UNC-37/Groucho in Caenorhabditis elegans cholinergic motor neurons,” J. Neurosci., 21:2001-2014, 2001.

Lin & Avery, Nature, 402:128-129, 1999.

Lincke, Broeks, Plasterk, Borst, “The expression of two P-glycoprotein (pgp) genes in transgenic Caenorhabditis elegans is confined to intestinal cells,” EMBO. J., 12:1615-1620, 1993.

Maduro and Pilgrim, “Identification and cloning of unc-119, a gene expressed in the Caenorhabditis elegans nervous system,” Genetics, 141:977-988, 1995.

Maniatis et al., Cell, 8:163, 1976.

Mattila et al., NAR, 19:4967-4973, 1991.

McClary et al., J. DNA Sequencing Mapping, 1(3):173-180, 1991.

Meinkoth and Wahl, Methods Enzymol., 152:91-94, 1987.

Miller and Niemeyer, “Expression of the unc-4 homeoprotein in Caenorhabditis elegans motor neurons specifies presynaptic input,” Development, 121:2877-2886, 1995.

Miller, Ortiz, Berliner, Epstein, “Differential localization of two myosins within nematode thick filaments,” Cell, 34:477-490, 1983.

Miller, Shen, Shamu, Burglin, Ruvkun, Dubois, Ghee, Wilson, “Caenorhabditis elegans unc-4 gene encodes a homeodomain protein that determines the pattern of synaptic input to specific motor neurons,” Nature, 355:841-845, 1992.

Miller, Stockdale, Karn, “Immunological identification of the genes encoding the four myosin heavy chain isoforms of Caenorhabditis elegans,” Proc. Natl. Acad. Sci. USA, 83:2305-2309, 1986.

Modrich & Richardson, J. Biol. Chem., 250:5515-5522, 1975.

Montgomery et al., Proc. Nat'l Acad. Sci. USA, 95:155-2-15507, 1998.

Murray and Kelley, Molec. Gen. Genet., 175:77-87, 1979.

Nelson and Riddle, “Functional study of the Caenorhabditis elegans secretory-exeretory system using laser microsurgery,” J. Exp. Zool., 231:45-56, 1984.

Nelson, Albert, Riddle, “Fine structure of the Caenorhabditis elegans secretory-excretory system,” J. Ultrastruct. Res., 82:156-171, 1983.

Nordstrom et al., J. Biol. Chem., 256:3112-3117, 1981.

Okkema, Ha, Haun, Chen, Fire, “The Caenorhabditis elegans NK-2 homeobox gene ceh-22 activates pharyngeal muscle gene expression in combination with pha-1 and is required for normal pharyngeal development,” Development, 124:3965-3973, 1997.

Perler et al., Adv. Protein Chem., 48:377-435, 1996

Perler et al., Proc. Nat'l Acad. Sci. USA, 89:5577, 1992.

Plasterk and Ketting, “The silence of the genes,” Curr. Opin. Genet. Dev., 10:562-567, 2000.

Richmond, Davis, Jorgensen, “UNIC-13 is required for synaptic vesicle fusion in Caenorhabditis elegans,” Nat. Neurosci, 2:959-964, 1999.

Sambrook et al., In: Molecular Cloning: A Laboratory Manual, second edition, Cold Spring Harbor Laboratory, Cold Spring Harbor, N.Y., 1989.

Sanger et al., Proc. Nat'l Acad. Sci. USA, 74:5463-5467, 1977.

Sharp & Zamore, Science, 287:2431-2433, 2000.

Sharp, Genes Dev., 13:139-141, 1999.

Singh and Sulston, “Some observations on molting in Caenorhabditis elegans,” Nematologica, 24:63-71, 1978.

Strange and Spring, “Absence of significant cellular dilution during ADH-stimulated water reabsorption,” Science, 235:1068-1070, 1987.

Strange and Spring, “Cell membrane water permeability of rabbit cortical collecting duct.,” J. Memb. Biol., 96:27-43, 1987.

Strange and Spring, “Methods for imaging renal tubule cells,” Kidney Intl., 30:192-200, 1986.

Studier et al., Methods Enzymol., 185:60-89, 1990.

Tabara et al., Cell, 99:123-132, 1999.

Tabor and Struhl, In: Current Protocols in Molecular Biology, Ausubel et al. (Eds.), John Wiley and Sons, NY, pp 3.5.10-3.5.12, 1989.

Tavernarakis, Wang, Dorovkov, Ryazanov, Driscoll, “Heritable and inducible genetic interference by double-stranded RNA encoded by transfenes,” Natl. Genet., 24:180-183, 2000.

Terns, Kroll-Conner, Zhu, Chung, Rothman, “A deficiency screen for zygotic loci required for establishment and patterning of the epidermis in Caenorhabditis elegans,” Genetics, 146:185-206, 1997.

Timmons, Court, Fire, “Ingestion of bacterially expressed dsRNAs can produce specific and potent genetic interference in Caenorhabditis elegans,” Gene, 263:103-112, 2001.

Ui-Tei, K., Zenno, S., Miyata, Y., and Saigo, K. “Sensitive assay of RNA interference in Drosophila and Chinese hamster cultured cells using firefly luciferase gene as target,” FEBS Lett. 479, 79-82, 2000.

Vukmirovic and Tilghman, Nature 405:820-822, 2000.

Wadsworth and Hedgecock, “guidance of neuroblast migrations and axonal projections in Caenorhabditis elegans,” Curr. Opin. Neurobiol., 2:36-41, 1992.

White, “The anatomy,” In: The Namatode Caenorhabditis elegans, Wood and the Community of Caenorhabditis elegans Researchers (Eds.), Cold Spring Harbor Press, 81-122, 1988.

Winnier, Meir, Ross, Tavernarakis, Dirscoll, Ishihara, Katsura, Miller, “UNC-4/UNC-37-dependent repression of motor neuron-specific genes controls synaptic choice in Caenorhabditis elegans,” Genes. Dev., 13:2774-2786, 1999. Adler & Modrich, J. Biol. Chem., 254:11605-11614, 1979.

What is claimed is:

1. A method for assigning functional properties to polypeptides comprising:
(a) obtaining a population of double-stranded (ds) RNA molecules, where each unique dsRNA molecule is segregated from other members of the dsRNA population;
(b) contacting one or more members of said dsRNA population with a host cell; and
(c) measuring one or more phenotypic parameters of the host cell of step (b),
wherein a change in a phenotypic parameter in the host cell of step (b), as compared to a host cell not contacted with dsRNA, assigns to a polypeptide or polypeptides encoded by the one or more members of said dsRNA population of step (b) a functional property.

2. The method of claim 1, wherein step (b) comprises contacting a first set of about 10 to about 100 different dsRNA sequences with said host cell.

3. The method of claim 2, further comprising:
(d) contacting each of the dsRNA sequences used in step (b) with a different host cell; and
(e) measuring one or more phenotypic parameters of each of the host cells of step (d),
wherein a change in a phenotypic parameter a host cell of step (d), as compared to a host cell not contacted with dsRNA, assigns to a polypeptide encoded by the dsRNA of step (d) a functional property.

4. The method of claim 1, further comprising the step, prior to step (a), of preparing dsRNA.

5. The method of claim 4, further comprising, prior to the step of preparing dsRNA, of preparing cDNA.

6. The method of claim 1, further comprising sequencing of one or more nucleic acids following assignment of a functional property.

7. The method of claim 1, wherein the host cell is bacterial, yeast, or mammalian.

8. The method of claim 1, wherein the phenotypic parameter is selected from the group consisting of cell growth, cell proliferation, apoptosis, Ca²⁺ signaling, ion channel activity, ion transporter activity, drug action, toxin action, metabolism, viral infectino, bacterial infection and stress response.

9. The method of claim 1, wherein said dsRNA population is derived from C. elegans, Drosophila or Arabidopsis.

10. The method of claim 2, wherein said method comprises concurrent testing of multiple sets of about 10 to about 100 dsRNA with individual host cells.

11. The method of claim 4, further comprising, prior to the step producing said dsRNA population, of performing a DNA or RNA subtraction with another population of DNA or RNA.

12. A method for identifying a functionally relevant polypeptides comprising:
(a) obtaining a population of double-stranded (ds) RNA molecules, where each unique dsRNA molecule is segregated from other members of the dsRNA population;
(b) contacting one or more members of said dsRNA population with a host cell; and
(c) measuring one or more phenotypic parameters of the host cell of step (b),
wherein a change in a phenotypic parameter in the host cell of step (b), as compared to a host cell not contacted with dsRNA, assigns to a polypeptide or polypeptides encoded by said one or more members of said dsRNA population of step (b) a relevant function.

13. A method for screening a population of nucleic acids for functionally relevant polypeptides comprising:
(a) obtaining a population of double-stranded (ds) RNA molecules, where each unique dsRNA molecule is segregated from other members of the dsRNA population;
(b) contacting one or more members of said dsRNA population with a host cell; and
(c) measuring one or more phenotypic parameters of the host cell of step (b),
wherein a change in a phenotypic parameter in the host cell of step (b), as compared to a host cell not contacted with dsRNA, assigns to a polypeptide or polypeptides encoded by said one or more members of said dsRNA population of step (b) a relevant function.
